(19) United States
(12) Patent Application Publication
(10) Pub. No.: US 2003/0008836 A1
(43) Pub. Date: Jan. 9, 2003

(54) Eukaryotic gene expression cassette and uses thereof

(75) Inventor: Geoffrey Goldspink, London (GB)

Correspondence Address: PENNIE AND EDMONDS, 1155 AVENUE OF THE AMERICAS, NEW YORK, NY, 100362711

(73) Assignee: University College London

(21) Appl. No.: 10/005,131

(22) Filed: Dec. 5, 2001

Related U.S. Application Data
(63) Continuation of application No. 09/403,707, filed on Mar. 17, 2000, now abandoned.

(30) Foreign Application Priority Data
Apr. 25, 1997 (GB) 9708526.0
Apr. 25, 1998 PCT/GB98/01198

(51) Int. Cl. A61K 48/00; C12N 9/24; C12N 15/85

(52) U.S. Cl. 514/44; 435/320.1; 435/455; 435/200; 424/93.2

(57) ABSTRACT

The present invention provides an expression cassette comprising, operably linked, (i) a myosin light chain enhancer, (ii) a promoter selected from a myosin heavy chain promoter and a viral promoter and (iii) a polynucleotide sequence of interest. The expression cassette can be used in methods of medical treatment and vaccination.

The present invention relates to a gene expression cassette. The expression cassette can be used for directing expression of heterologous genes in eukaryotic cells. It also relates to the use of said expression cassette in gene therapy and vaccine production. It further relates to vectors, including viral strains, comprising said expression cassette.

### BACKGROUND TO THE INVENTION

Anderson-Fabry disease is a lysosomal storage disorder (LSD) resulting from the deficiency of the lysosomal enzyme alpha-galactosidase (alpha-gal, EC 3.2.1.22). This enzymatic defect leads to the deposition of neutral glycosphingolipids in most tissues, the pathological and clinical manifestations of the disease being the result of progressive accumulation in endothelial cells leading to ischemia and infarction in organs like kidney, heart or brain.

In addition to the sorting mechanisms operating in the trans-Golgi network, lysosomal enzymes can also be recaptured from the extracellular space via mannose-6-phosphate receptors. In keeping with this, it has been shown that the administration of purified lysosomal enzymes to the culture medium can correct the enzymatic defect in fibroblasts from patients with various types of LSD. This ability of cells to take up the enzyme has provided the basis for the use of replacement therapy for this group of disorders. In the case of Fabry disease, early studies showed that alpha-gal partially purified from various sources is taken-up by skin fibroblasts from Fabry hemizygotes when added to the culture medium and does catabolize the accumulated substrate, globotriaosylceramide (CTH). This prompted several clinical trials of enzyme replacement in the 1970s which demonstrated the feasibility of enzyme therapy for Fabry disease. However, the unavailability of sufficient amounts of the purified human enzyme has prevented a proper evaluation of the efficacy of replacement therapy so far.

Alternative ways of providing a source of active enzyme for the treatment of LSD have included bone marrow transplantation and, more recently, gene transfer into haematopoietic stem cells or enzyme delivery into the whole organism by genetically modified cells. For instance, it has been recently shown that fibroblasts transfected with retroviral vectors and grown on collagen lattices which were implanted in the peritoneal cavity successfully secreted beta-glucuronidase and corrected the storage lesions in the liver and spleen of Mucopolysacharidosis VII mice. The same approach resulted in long-term secretion of this enzyme in dogs and similar results were obtained in nude mice transplanted with neo-organs which were secreting alpha-L-iduronidase.

Since the discovery that skeletal muscle can be transfected in vivo by intramuscular injection of plasmid DNA, this organ system has attracted considerable attention as a potential source of secreted therapeutic proteins. Injection of plasmid DNA constructs has been used successfully for the expression of dystrophin, factor VII, apolipoprotein-E and alpha-1 antitrypsin, whereas intramuscular injection of genetically modified myoblasts gave encouraging results in the secretion of human growth hormone, factor IX, beta-glucuronidase, human and murine erythropoieiin and human glucocerebrosidase. However, the efficiency of these methods of transfection is still low, even with the induction of muscle degeneration and regeneration through injection of myotoxic substances prior to the injection of DNA. Direct plasmid injection in muscle shows better transfection efficiency than viral vectors and plasmid DNA has been found to be maintained extrachromosomally for at least 19 months. Moreover, the safety, simplicity and low-cost of intramuscular injection of plasmid DNA make it a very attractive alternative to other methods. However, most studies so far have shown that expression is not high enough to increase the blood levels of circulating proteins.

### SUMMARY OF THE INVENTION

The present invention relates to an expression cassette comprising, operably linked, (i) a myosin light chain enhancer, (ii) a promoter selected from a myosin heavy chain promoter and a viral promoter and (iii) a polynucleotide sequence of interest.

Nucleic acid constructs, including virus strains, comprising said expression cassette can be used, for example, for delivering therapeutic genes in methods of treatment of diseases, for example Fabry disease, or for the delivery of genes encoding specific antigens for vaccine purposes.

Accordingly the present invention provides an expression cassette comprising, operably linked, (i) a myosin light chain enhancer, (ii) a promoter selected from a myosin heavy chain promoter and a viral promoter and (iii) a polynucleotide sequence of interest.

Preferably, the enhancer is a myosin light chain 1/3 enhancer. Preferably the myosin heavy chain promoter is a mammalian heavy chain promoter, more preferably a truncated rabbit-myosin heavy chain promoter. Preferably the viral promoter is a cytomegalovirus (CMV) or herpes simplex virus (HSV) promoter.

The expression cassette of the invention may thus be used to deliver a polynucleotide sequence of interest to a eukaryotic cell where it A will be expressed. Vectors and viral strains comprising the expression cassette of the invention may also be used to deliver a polynucleotide sequence of interest to a eukaryotic cell where it will be expressed. Preferably the cell is a vertebrate cell, more preferably an avian, fish or mammalian muscle cell. Such expression cassettes, vectors and viral strains are useful in a variety of applications, for example, in methods of medical treatment including gene therapy and as vaccines.

Preferably, the polynucleotide sequence of interest comprises a heterologous gene. The heterologous gene may be any allelic variant of a wild-type gene, or it may be a mutant gene. The heterologous gene preferably encodes a polypeptide of therapeutic use.

The invention further provides for the use of the expression cassette, vectors and viral strains, comprising the expression cassette, for use in the treatment of humans and animals.

The invention also provides a method for producing a viral strain comprising an expression cassette of the invention, which method comprises introducing an expression cassette of the invention into the genome of the virus strain, preferably by homologous recombination.

### DETAILED DESCRIPTION OF THE INVENTION

A. Expression Cassette—myosin light chain enhancer, myosin heavy chain promoter/viral promoter, polynucleotide sequences of interest.

The expression cassette of the invention comprises, operably linked, (i) a myosin light chain enhancer, (ii) a promoter selected from a myosin heavy chain promoter and a viral promoter and (iii) a polynucleotide sequence of interest. The term “operably linked” refers to a juxtaposition wherein the components are in a relationship permitting them to function in their intended manner. Thus, for example, a promoter operably linked to a polynucleotide sequence of interest is ligated in such a way that expression of the polynucleotide sequence of interest is achieved under conditions which are compatible with the activation of expression from the promoter.

The expression cassette can be constructed using routine cloning techniques known to persons skilled in the art (see, for example, Sambrook et al., 1989, Molecular Cloning—a laboratory manual; Cold Spring Harbor Press).

### 2. Myosin Enhancer

Several myosin enhancers have been identified to date from both myosin light chain and myosin heavy chain genes. Preferably the enhancer used in the expression cassette of the present invention is of vertebrate origin, more preferably avian, piscine or mammalian origin. A myosin light chain enhancer is preferred. A rat myosin light chain 1/3 enhancer (Donoghue et al., 1988 ; Neville et al., 1996), is especially preferred. The enhancer is operably linked to the promoter. The term ‘operably linked’ is as defined above. The enhancer may be either upstream or downstream of the promoter. The enhancer may be used in either orientation.

### 3. Promoters

The promoter in the expression cassette of the invention is selected from myosin heavy chain promoters or viral promoters which are functional in vertebrate cells, preferably avian, piscine and/or mammalian, preferably human, cells. The myosin heavy chain promoter is preferably a truncated rabbit β-cardiac myosin heavy chain promoter, in particular up to and including 789 base pairs upstream of the transcription start site. Another myosin heavy chain promoter which is especially preferred is the carp FG2 promoter, in particular up to and including 901 base pairs upstream of the transcription start site (Gauvry et al., 1996). Further details of myosin heavy chain promoters derived from rat, rabbit, human, porcine and chick myosin heavy chain genes are given in Gauvry et al. . 1996 and references therein. Viral promoters include CMV and HSV promoters. CMV IE promoters are especially preferred.

### 4. Polynucleotide Sequences of Interest

The term “polynucleotide sequence of interest” is intended to cover nucleic acid sequences which are capable of being at least transcribed. The sequences may be in the sense or antisense orientation with respect to the promoter. Antisense constructs can be used to inhibit the expression of a gene in a cell according to well-known techniques. The polynucleotide sequence of interest may comprise a heterologous gene. The term heterologous gene encompasses any gene. Thus sequences encoding mRNA, tRNA and rRNA are included within this definition. The heterologous gene ma y be any allelic variant of a wild-type gene, or it may be a mutant gene. Sequences encoding mRNA will optionally include some or all of 5′ and/or 3′ transcribed but untranslated flanking sequences naturally, or otherwise, associated with the translated coding sequence. The polynucleotide sequence of interest may optionally further include the associated transcriptional control sequences normally associated with the transcribed sequences, for example transcriptional stop signals, polyadenylation sites and downstream enhancer elements.

The polynucleotide sequence of interest preferably encodes a therapeutic product, which can for example be a peptide, polypeptide, protein or ribonucleic acid. More especially, the coding sequence is a DNA sequence (such as cDNA or genomic DNA) coding for a polypeptide product such as enzymes , (e.g. β-galactosidase), blood derivatives, hormones, cytokines, namely interleukins, interferons or TNF, growth factors (e.g. IGF-1), neurotransmitters or their precursors or synthetic enzymes, trophic factors such as BDNF, CNTF, NGF, IGF, GMF, aFGF, bFGF, NT3 and NT5; apolipoproteins. such as ApoAI, ApoAIV and, dystrophin or a minidystrophin, tumour-suppressing genes such as p53, Rb, Rap1A DCC and k-rev, genes coding for factors involved in coagulation such as factors VII, VIII and IX or alternatively all or part of a natural or artificial immunoglobulin (e.g. Fab and ScFv).

The coding sequence can also be an antisense sequence, whose expression in the target cell enables gene expression or the transcription of cellular mRNAs to be controlled. Such sequences can, for example, be transcribed in the target cell into RNAs complementary to cellular mRNAs and can thus block their translation into protein, according to the technique described in European Patent No. 140,308. In particular, antisense sequences can be used to block translation of inflammatory or catebolic cytokines in the treatment of arthritis and tissue loss caused by these cytokines.

The present invention may also be used for the expression of sequences coding for toxic factors. The latter can be, in particular, cell poisons (such as diphtheria toxin, pseudomonas toxin and ricin A), a product inducing sensitivity to an external agent (suicide genes: e.g. thymidine kinase and cytosine deaminase) or alternatively killer genes capable of inducing cell death (e.g. Grb3-3 and anti-ras ScFv).

Preferably, the polynucleotide sequence of interest encodes a polypeptide of therapeutic use. For example, of the proteins described above, α-galactosidase can be used to treat Fabry disease.

Polynucleotide sequences of interest may also encode antigenic polypeptides or nucleic acids for use as vaccines. Preferably such antigenic polypeptides or nucleic acids are derived from pathogenic organisms, for example bacteria or viruses. For example, antigenic polypeptides or nucleic acids may be selected from regions of the hepatitis C virus genome and gene products. Antigenic determinants present in the genomes or gene products of the causative agents of, for example, viral haemorrhagic septicemia, bacterial kidney disease, vibriosis and furunculosis are particularly preferred.

Heterologous genes may also include marker genes (for example encoding—galactosidase or green fluorescent protein) or genes whose products regulate the expression of other genes.

### B. Vectors

The expression cassette may be used in the form of a naked nucleic acid construct. Alternatively, it may be introduced into a variety of nucleic acid vectors. Such vectors include plasmids and viral vectors. Vectors may further include sequences flanking the expression cassette which comprise sequences homologous to eukaryotic genomic sequences, preferably mammalian genomic sequences, or viral genomic sequences. This will allow the introduction of the expression cassette into the genome of eukaryotic cells or viruses by homologous recombination. In particular, a plasmid vector comprising the expression cassette flanked by viral sequences can be used to prepare a viral vector suitable for delivering the expression cassette to a vertebrate, including fish, avian or mammalian, cell. The techniques employed are well-known to a skilled person.

### D. Administration

The expression cassette of the invention may thus be used to deliver therapeutic genes to a human or animal in need of treatment. Alternatively, the expression cassette of the invention may be used to deliver genes encoding potentially immunogenic polypeptides in vivo for vaccine purposes particularly the vaccination of fish.

The expression cassette of the invention may be administered directly as a naked nucleic acid construct, preferably further comprising flanking sequences homologous to the host cell genome. Uptake of naked nucleic acid constructs by vertebrate cells is enhanced by several known techniques including biolistic transformation and lipofection.

Alternatively, the expression cassette may be administered as part of a nucleic acid vector, including a plasmid vector or viral vector.

Preferably the delivery vehicle (i.e. naked nucleic acid construct or viral vector comprising the expression cassette for example) is combined with a pharmaceutically acceptable carrier or diluent to produce a pharmaceutical composition. Suitable carriers and diluents include isotonic saline solutions, for example phosphate-buffered saline. The composition is typically formulated for intramuscular administration.

Preferably, the substance is used in an injectable form. It may therefore be mixed with any vehicle which is pharmaceutically acceptable for an injectable formulation, preferably for a direct injection at the site to be treated. The pharmaceutically carrier or diluent may be, for example, sterile or isotonic solutions. It is also preferred to formulate that substance in an orally active form. Methods for injecting nucleic acids into fish muscle are described in Gauvry et al., 1996.

The actual formulation used can be readily determined by the skilled person and will vary depending on the nature of the substance to be administered and the route of administration.

The dose of substance used may be adjusted according to various parameters, especially according to the substance used, the age, weight and condition of the patient to be treated, the mode of administration used and the required clinical regimen. A physician will be able to determine the required route of administration and dosage for any particular patient and condition.

The invention will be described with reference to the following Example which are intended to be illustrative only and not limiting.

### EXAMPLE 1

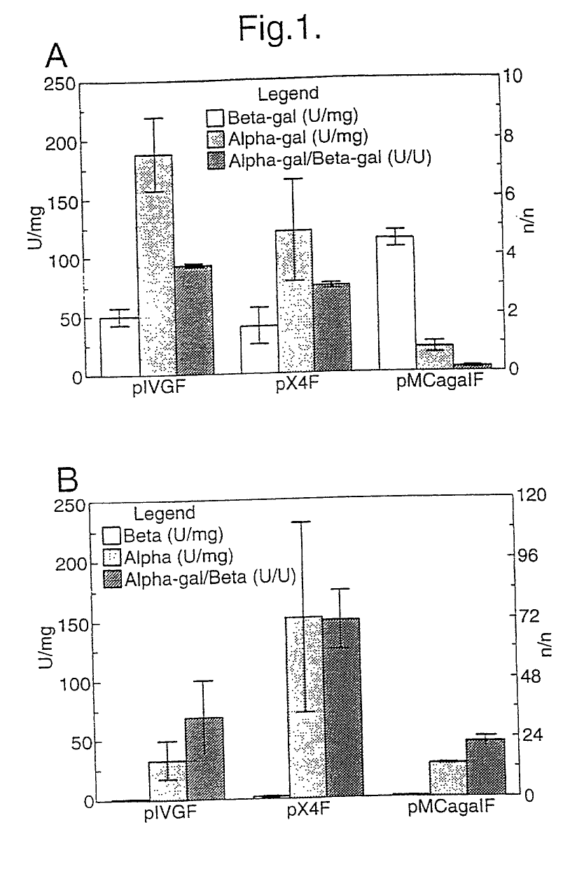

### BRIEF DESCRIPTION OF THE FIGS. 1 to 5

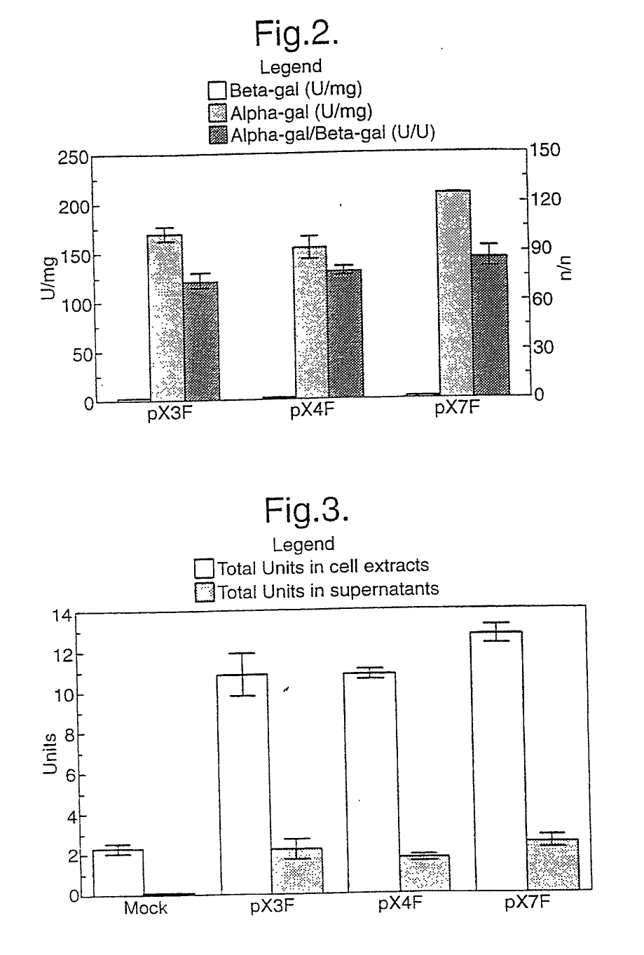

FIG. 1 and FIG. 2 are graphs showing a comparison of alpha-gal and beta-gal activity obtained using three different constructs.

FIG. 3 is a graph showing alpha-gal activity in the cell extracts/supernatants obtained from myoblasts transfected with three different constructs.

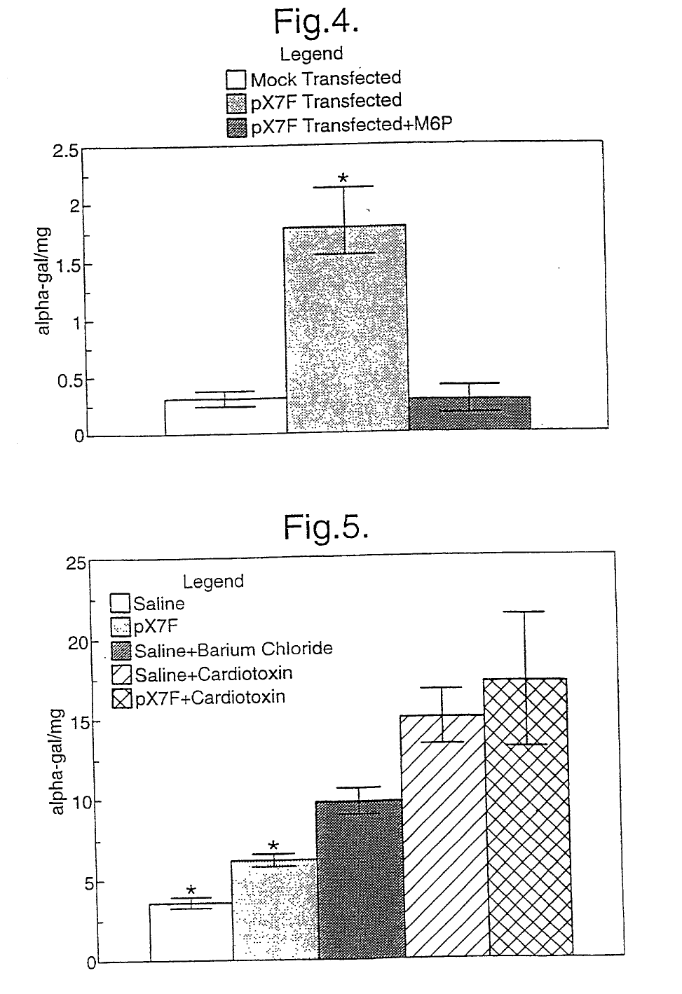

FIG. 4 is a graph showing alpha-gal activity in cell extracts from fibroblasts from a Fabry patient which have been transfected with an alpha-gal expressing construct.

FIG. 5 is a graph showing alpha-gal activity in muscle extracts 7 days after injection with an alpha-gal expressing construct.

### DETAILED DESCRIPTION OF THE FIGS. 1 to 5

FIG. 1:

A. Comparison of constructs pIVGF, pX4F and pMCagalF after transfection of C2C12 myoblasts (see Table 1 for details of constructs). DNA for transfections was prepared using Plasmid midi-columns (Qiagen, Dorkin g, UK). C2C12 mouse myoblasts were plated at 1.5×10⁴ cells/cm² and grown overnight in growth medium (DMEM N10%FCS with penicillin-streptomycin-amphotericin B). Transfections were performed mixing 10 g of Lipofectamine (Gibco, Paisley, UK) with 2 μg of DNA in 200 μl of Optimem-1 (Gibco, Paisley, UK). After 30 min incubation at room temperature, the mixture was diluted up to 1 ml in Optimem-1 and added to the cells. Transfections were carried out for 6-8 hours at 37° C./5% CO₂ and included pCMV-b (typically 200 ng in 2 μg of total DNA) which drives the expression of beta-galactosidase. The latter was used as an internal control of transfection efficiency. After transfection, plates were washed with PBS followed by addition of DMEM/2% horse serum (differentiation medium). Under these conditions, myoblasts start the process of fusion and differentiation into myotubes, which become visible after 48 hours and continue to develop for 4-6 days. Enzvmatic activities of alpha-galactosidase and beta-galactosidase in cell extracts were assayed fluorimetrically with specific substrates, so that both reactions do not show any cross-reactivity. Normalized alpha-gal enzymatic activity (in Units alpha-gal/Unit beta-gal) is shown 18 hours post-transfection (undifferentiated myoblasts). High-Low bars show the results from duplicate experiments.

B. Comparison of constructs pIVGF, pX4F and pMCagalF after transfection of C2C12 myoblasts as indicated above. Normalized alpha-gal enzymatic activity (in Units alpha-gal/Unit beta-gal) is shown 10 days post-transfection (fully differentiated myotubes). Note that the values reflect not only the change in alpha-gal but also the decrease in beta-gal (the reporter enzyme used to correct for transfection efficiency), which is driven by the CMV promoter alone. Therefore, the actual normalized units for each construct cannot be compared directly with the values obtained from undifferentiated myoblasts (shown in FIG. 1A). However, since all constructs are co-transfected with exactly the same amount of the same internal control plasmid, a direct comparison can be made between constructs at a given time point post-transfection. High-Low bars show the results from duplicate experiments.

FIG. 2: Comparison of constructs pX3 F, pX4F and pX7F after transfection of C2C12 myoblasts as indicated in FIG. 1. Enzymatic activity of beta-gal and alpha-gal (in Units/mg of protein, left axis) and the normalized alpha-gal enzymatic activity (in Units alpha-gal /Unit beta-gal) 48 hours post-transfection (small myotubes) are shown. High-Low bars show the results from duplicate experiments.

FIG. 3: Total alpha-gal activity (in Units, 1 Unit=1 nmol/h) of cell extracts and of supernatants from C2C12 myoblasts transfected with three different constructs (Mock—no DNA transfected) and harvested 48 hours after transfection. Total alpha-gal actvity was derived from the orginal enzymatic activity in cell extracts (in Units/mg) or in supernatants (in Units/L). High-low bars show the results from duplicate experiments.

FIG. 4: Alpha-gal activity in cell extracts from fibroblasts of a Fabry patient that were cultured for 4 days in medium conditioned by C2C12 myoblasts transfected as indicated, either in the absence or in the presence (+M6P) of 5 mM mannose-6-phosphate (Sigma, Poole, UK) in the culture medium. Conditioned media were 0.22 mm-filtered before being added to the fibroblasts in order to avoid carry-over of the liposome-DNA complex. Proteins were measured using the bicinchonic acid method (Sigma, Poole, UK).

Error bars=S.E.M. (n=6).

*Significant difference (p<0.01) with any of the other groups (Mann-Whitney ranks-sum test for unpaired samples).

FIG. 5: Alpha-gal activity (in Units/mg of protein) in tibialis anterior muscle extracts 7 days after injection. DNA of construct pX7F was prepared using the Endo-free Plasmid Kit (Qiagen, Dorking, UK). 30 mg of DNA in 50 ml of sterile, endotoxin-free saline (or 50 ml of saline in control muscles) were injected in tibialis anterior muscles of 5-6 week-old C57B1/6 mice following previous recommendations³¹. Mice were anaesthetized with Hypnorm-Diazepam and the DNA solution was injected percutaneously in the centre of the muscle with a tuberculin syringe fitted with a 27G needle, using a perpendicular approach.

Seven days after injection the animals were sacrificed and the muscles were dissected and frozen at −70° C., finely ground on a pre-cooled mortar and then vortexed for 15 min at room temperature in 500 ml of Reporter Lysis Buffer (Promega, Southampton, UK), spun for 3 min. at 4° C. and the supernatants stored at −70° C. Proteins and alpha-gal enzymatic activity were determined as described above, and results were expressed in Units/mg of protein.

Some muscles were pre-injected with myotoxic substances (1.2% BaCl₂ or 0.1M cardiotoxin from Naja nigricollis) 5 days prior to the injection of DNA, in order to induce a cycle of degeneration/regeneration.

Error bars=S.E.M. (n=6).

p<0.01 between these two groups (Mann-Whitney ranks-sum test for unpaired samples).

Effect of Muscle-specific Regulatory Elements

We have made several vectors driving the expression of human alpha-gal and we have compared them following transfection of the myogenic cell line C2C12. This cell line has been extensively used as a model of muscle differentiation in the study of muscle-specific regulatory elements, due to the ability of these myoblasts to undergo fusion and differentiation under certain culture conditions. Once mature myotubes are formed, they cannot be transfected by non-viral methods. Therefore, the standard transfection protocol starts with transfection of undifferentiated myoblasts with cationic liposomes followed by differentiation to mature myotubes. Gene expression can be measured at any time point during the differentiation process, so that this system also allows a comparison of the activity of regulatory elements before and after the activation of muscle-specific genes. However, transfection efficiency must be carefully controlled by co-transfection with a different plasmid driving the expression of a reporter gene. This is used as an internal control which corrects for the differences between constructs and between plates.

We first compared three constructs containing a muscle-specific promoter (rabbit myosin heavy chain) or the human cytomegalovirus promoter combined with the myosin light chain 1/3 enhancer (see Table I for details of the constructs). The MLC1/3 enhancer has been shown to result in muscle-specific expression of heterologous genes in transgenic mice and zebrafish.

The construct in which alpha-gal expression is driven by the CMV promoter alone (pIVGF) showed the highest activity in undifferentiated myoblasts (18 hours post-transfection), followed by pX4F and pMCagalF in this order (FIG. 1A). In contrast, the analysis of fully differentiated myotubes (10 days post-transfection) revealed that alpha-gal enzymatic activity was clearly higher in pX4F than in pIVGF (FIG. 1B), the only difference between both constructs being the presence of the MLC1/3 enhancer in pX4F. Our results show that this enhancer element can increase the strength of the expression driven by the CMV promoter in differentiated myogenic cells, but exerts little or no enhancing effect in undifferentiated myoblasts. We have also compared the expression levels generated by pX3F and pX4F, which only differ in the orientation of the MLC1/3 enhancer. Both constructs showed the same activity after 6 days in differentiation medium (FIG. 2), confirming that the orientation of the enhancer does not affect the levels of expression. This supports previous data showing that this enhancer increases the activity of the SV40 promoter in mature myotubes in an orientation-independent manner and suggests that muscle differentiation provides the necessary muscle-specific factors which enable this element to enhance the basal activity of heterologous promoters.

TABLE 1

| CONSTRUCT | PARENT VECTOR | PROMOTER | ENHANCER |
|---|---|---|---|
| PMCagalF | pbPASe9 | MHC | MLC1/3 |
| PIVGF | pcDNA3 | CMV | — |
| pX3F | pcDNA3 | CMV | MLC1/3 (sense) |
| pX4F | pcDNA3 | CMV | MLC1/3 |
| | | | (antisense) |
| pX7F | pcDNA3 | CMV | MLC1/3 (sense) |

*pcDNA3 (Invitrogen, DeSchelp, The Netherlands)

Table 1: Details of the expression vectors used in this study. Rabbit β-cardiac myosin heavy chain (MHC) promoter consists of 781 bases of the promoter region. Myosin light chain 1/3 enhancer (MLC1/3) has accession number X14726. CMV is the major intermediate early promoter/enhancer region of human cytome galovirus. Constructs pX3F, pX4F and pX7F contain the MLC1/3 enhancer cloned either in the direction of transcription (sense) or in the reverse orientation (antisense) as indicated. For the generation of pX3F, pX4F and pIVGF, the cDNA coding for alpha-galactosidase was amplified by RT-PCR and cloned in pCRII™ (Invitrogen, DeSchelp, The Netherlands), resulting in pGal-wt. EcoRI digestion of pGal-wt releases the alpha-gal cDNA without flanking sequences which was used in the appropriate vectors. For the construction of pX7F we used a different fragment containing the cDNA for alpha-gal (gift from Dr. H. Sakuraba) which only contains 25 bp of 5′-UTR and no flanking sequences. Thus, pX3F and pX7F differ only in the length of the 5′-UTR of alpha-gal (35 bp longer in pX3F).

Secretion of Human Alpha-gal to the Culture Medium and Uptake by Alpha-gal-deficient Fibroblasts

In a different set of experiments, we have assayed the total activity of alpha-gal in cell extracts and in supernatants conditioned for 48 hours after transfection with constructs pX3F, pX4F or pX7F, relative to controls (mock transfected, no DNA). The total amount of alpha-gal activity in the culture medium of transfected cells was significantly higher than in controls (average of the three constructs=2.17 Units vs. 0.10 Units in controls, results not shown). In order to confirm that the alpha-gal expressed and secreted in vitro has undergone correct post-translational processing, we investigated the ability of alpha-gal-deficient fibroblasts to take up the enzyme from medium conditioned by C2C12 myoblasts transfected with pX7F. Fibroblasts from a hemizygous Fabry patient (which show low enzymatic activity) were cultured for 4 days in differentiation medium that had been conditioned by C2C12 myoblasts transfected with pX7F. FIG. 3 shows the alpha-gal enzymatic activity in fibroblasts kept under these conditions; significantly higher levels were detected in those cultured with medium conditioned by pX7F-transfected myoblasts than in those cultured with medium conditioned by mock-transfected myoblasts (p<0.01). This effect was completely abolished by the addition of mannose-6-phosphate (5 mM) to the conditioned medium (FIG. 3), showing that this increase in alpha-gal activity was the result of uptake of the enzyme via mannose-6-phosphate receptors. This strongly suggests that the enzyme contained appropriate post-translational modifications, like phosphomannosyl residues. To our knowledge, this is the first report in which a correctly glycosylated form of human alpha-gal was expressed and secreted from differentiated muscle cells.

Production of Human Alpha-gal After Intramuscular Injection of Plasmid DNA

We have analysed alpha-gal activity in muscle extracts from mice injected intramuscularly with plasmid DNA to see whether we could reproduce these results in vivo. This technique does not permit a correction to be made for the number of transfected fibres as can be done in vitro. This requires the use of samples of adequate size in order to detect any potential statistical difference. In our experiment (6 muscles in each group), injection of construct pX7F into tibialis anterior resulted in significantly increased levels of alpha-gal activity with respect to the control muscles injected with saline (p<0.01) 7 days after injection (FIG. 5). Expression of foreign proteins in muscle has been shown to elicit an immune response against those fibres expressing the protein, which decrease in number and disappear completely two weeks post-injection. For this reason, we have allowed expression to proceed only for one week, analysing enzymatic activity well before the start of the effector phase of the immune response against human alpha-gal. Preliminary results from our laboratory indicate that vectors containing the mouse alpha-gal cDNA can drive the expression of the enzyme for at least four weeks in injected muscles (not shown). The isogenic protein products of therapeutic genes can also trigger an immune response following their delivery in gene therapy. Therefore, measures to prevent or to combat immunogenicity of the targeted protein should be developed. Administration of several different immunomodulating agents, including immunosuppressive drugs and anti-CD4 or anti-CD40L antibodies at the time of gene therapy gave encouraging results. Such a transient ablation of CD4⁺ T cells may prevent the effector phase of immune response to the gene product. Moreover, some Fabry disease patients have missense mutations, possibly with some non-functional protein present. In these cases induction of immune responses to the gene therapy product may be prevented.

Apart from the immune reaction against transfected fibres, other authors have shown that the activity of the CMV-promoter driven expression declines after injection of plasmid constructs with reporter genes. In contrast, vectors containing the RSV promoter showed more sustained expression. The reasons for this difference between promoters is not well understood, but promoter shut-off has been proposed as a mechanism. However, our results suggest that the MLC1/3 enhancer element can increase and prolong the expression driven by the CMV promoter in conditions that resemble mature muscle fibres. This will have interesting implications for the development of vectors designed for direct plasmid injection in muscle, because it could help to maintain the activity of the CMV promoter for a longer period of time.

In summary, our in vivo results show significantly increased production of alpha-gal in muscle after injection of a plasmid expression vector. Together with our data showing that muscle cells can secrete human alpha-gal in its correctly processed form, the results presented here represent a significant step towards the improvement of this expression system and the generation of a strategy for the production of alpha-gal from muscle in vivo.

Transfer of the α-galactosidase Gene into a Mouse Model of Fabry's Disease

Knockout mice in which the α-galactosidase gene had been rendered non-functional were obtained from the National Institutes of Health (USA). These mice are therefore a model for Fabry's disease in humans because the deficiency underlying Fabry's disease is a deficiency in α-galactosidase, which enzyme is either absent or produced at inadequate levels (Ohshima, T. et al. (1977)).

Using constructs of the invention, (pX61 and pX62 containing CMV promoter, MLC 1/3 enhancer as with pX3F, pX4F and pX7F but containing a kanamycin resistance marker in place of the ampicillin resistance marker used in the latter), the α-galactosidase gene was introduced into these mice by known techniques. As described above, the constructs express the α-galactosidase gene under the control of the CMV promoter in combination with the rat myosin light chain 1/3 enhancer. Various combinations of ages and sexes of mice and sites of injection were tested and α-galactosidase activity in the muscles of the mice was assayed after one week or three weeks. The most striking results were obtained when the constructs were delivered to young animals by intramuscular injection and α-galactosidase levels were assayed after one week.

The results are given below. Numbers represent units of α-galactosidase activity in muscle per mg protein. (m) and (f) denote male and female mice. For reference, the levels of α-galactosidase activity it normal mice is in the region of 4-5 units. (The controls in the table have sub-normal levels because the α-galactosidase gene has been specifically knocked out.)

| | Activity |
|---|---|
| Control mice | −0.077 |
| | 0.21 |
| | −0.058 |
| | 0.075 |
| Subject mice | 143.906 (m) |
| | 628.536 (m) |
| | 96.442 (f) |
| | 706.214 (f) |
| | 212.93 (f) |

### REFERENCES

Gauvry L., Ennion S., Hansen, E. Butterworth P. and Goldspink G. (1996) The characterisation of the 5′ regulatory region of a temperature-induced myosin heavy chain gene associated with myotomal muscle growth in carp. Eur. J. Biochem., 236, 887-894

Donoghue M., Ernst H., Wentworth B., Nadal Ginard G. and Rosenthal N. (1988) A muscle specific enhancer is located at the 3′ end of the myosin light chain 1/3 gene locus. Genes Dev., 2, 1779-1790.

Neville C., Gonzales D., Houghton, L., McGrew M and Rosenthal N. (1996) Modular elements of the MLC 1f/3f locus confer fiber-specific transcription regulation in trangenic mice. Dev. Genetics, 19, 157-162.

Rosenthal N., Berglund E., Wentworth B, Donoghue M., Winter B., Braun, Bober E. and Arnold H. (1989) A highly conserved enhancer downstream of the human MLC 1/3 locus is a target for multiple myogenic factors. Nucleic Acids Res., 18, 6239-6245.

OhshimaT., et al., (1977), α-Galactosidase A deficient mice: A model of Fabry disease. Proc. Natl. Acad. Sci. USA., 94, 2540-2544.

### EXAMPLE 2

We tested the levels of expression achieved by a number of muscle specific promoters and a myosin light chain enhancer when spliced to the reporter gene chloramphenicol acetyltransferase (CAT), in vitro and in vivo by injection into fast and slow muscles of the mouse. The results show that the highest levels of expression are achieved by a combination of a truncated myosin heavy chain promoter and the enhancer, and that a whole range of expression levels is obtained with the other combinations tested. The data shows that a cassette based on these elements should provide efficient vectors for the introduction and expression of genes following intramuscular injection of naked DNA.

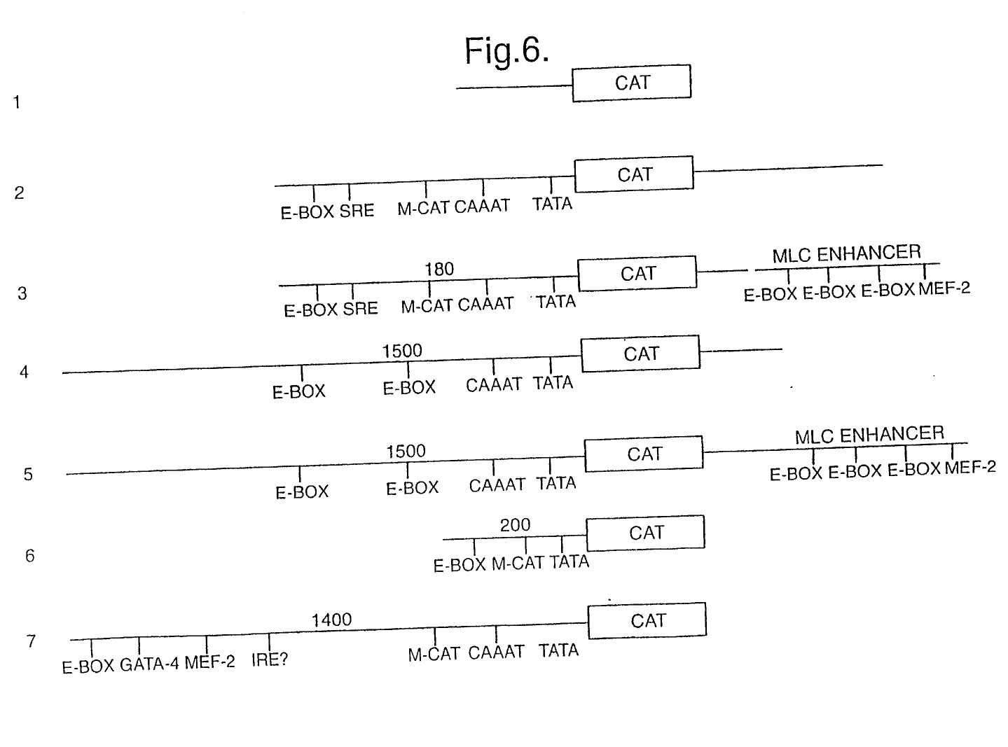

### DESCRIPTION OF FIGS. 6 to 9

FIG. 6. Schematic representation of the different muscle specific promoter fragments. The main muscle specific elements are shown in each drawing. Key: 1. SV-40-CAT, 2. MHC-CAT, rabbit β-myosin heavy chain fragment plus myosin light chain enhancer. 3 as 2 without the enhancer. 4. MLC1-CAT, rat myosin light chain promoter, plus myosin light chain enhancer 5. as 4 plus enhancer. 6. MCK-CAT, muscle specific fragment of M-CK promoter. 7. HMHC-CAT, human β-myosin heavy chain fragment.

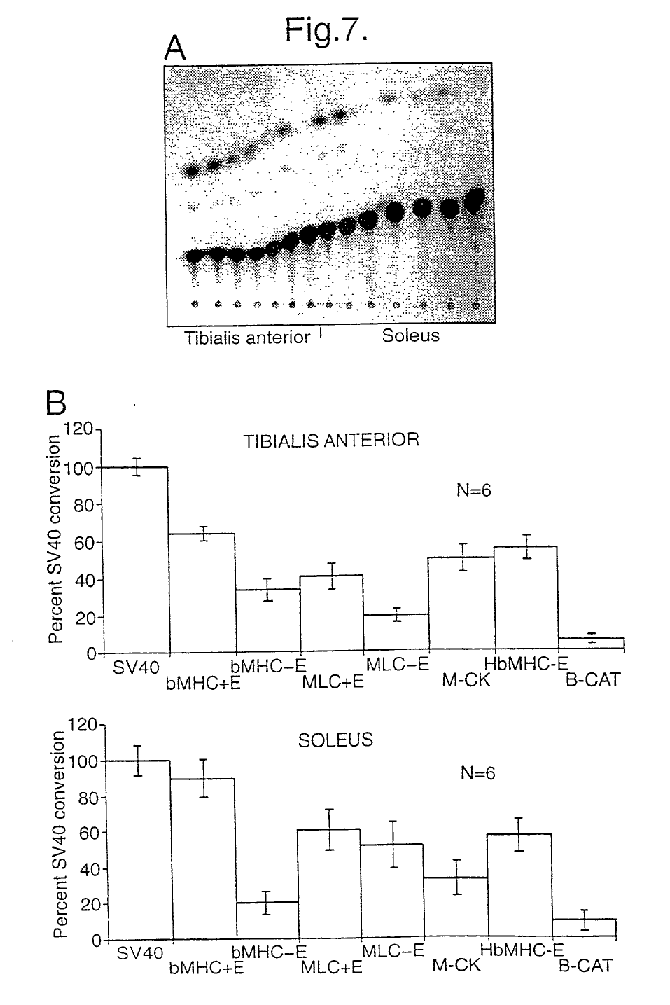

FIG. 7. Expression achieved by different muscle specific promoter fragments in C2C12 cells. Quantitation of CAT activity after C2C12 cell transfection with test constructs and the β-galactosidase containing plasmid (see Methods). Cell extract corresponding to 0.5 absorbance unit of β-galactosidase activity was assayed. The results represent the mean and standard deviation of four dishes. The order of constructs is as in FIG. 6.

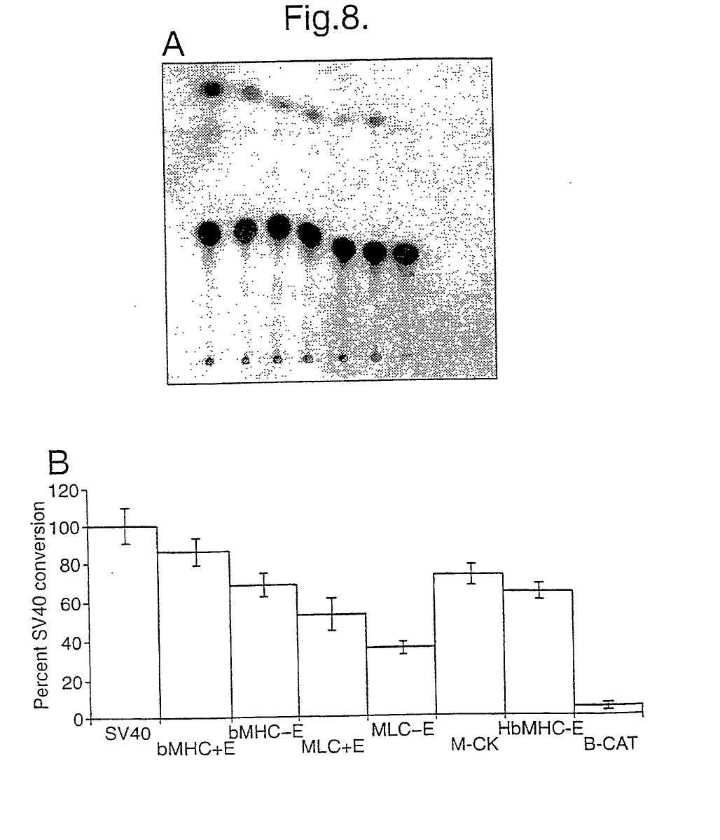

FIG. 8. Expression achieved by muscle specific promoter fragments in the tibialis anterior and soleus of normal mice. For each test construct, six muscles were injected. Cell extract corresponding to 0.5 absorbance unit of β-galactosidase (tibialis anterior) and 0.2 units (soleus) was assayed. The order of constructs is as in FIG. 6.

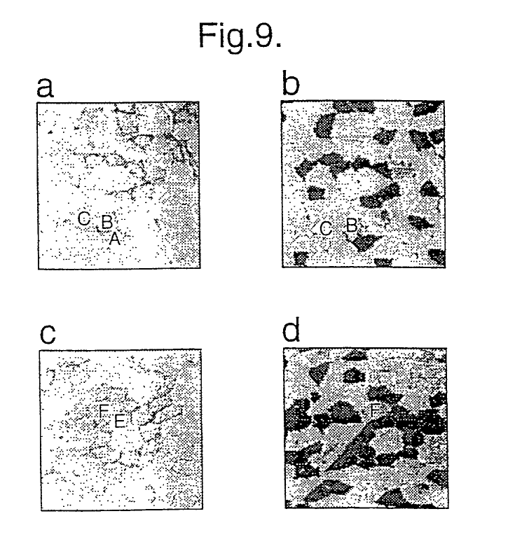

FIG. 9. Expression of the truncated rabbit myosin heavy chain construct in tibialis anterior muscle with or without enhancer. (a,c) anti-CAT staining, (b,d) myosin ATPase, alkaline preincubation. Myosin heavy chain promoter without enhancer (a,b). Staining of fast (A), intermediate (B) and slow fibres (C) is obtained. Myosin heavy chain promoter with myosin light chain enhancer (c,d). Fast (E) fibres are predominantly stained with some staining observed on intermediate fibres.

Transcriptional activity of muscle specific promoters in C2C12 cells. A number of promoter fragments were subcloned into CAT basic plasmid. In some cases a 900 bp myosin light chain enhancer was inserted in the BamH1 site of the plasmid, downstream from CAT. The constructs tested were: βMHC-CAT (βMHC) a 780 bp fragment of the rabbit β cardiac myosin heavy chain previously shown to be expressed specifically in skeletal muscle⁷ a 1500 bp fragment of myosin light chain 1/3f promoter⁸ a 1400 bp fragment of human β myosin heavy chain⁹ and a 200 bp promoter/enhancer fragment of M-CK, the muscle specific form of creatine kinase¹⁰ (FIG. 6). The rabbit β cardiac myosin heavy chain and the light chain promoter fragments was also tested with the myosin light chain enhancer. The construct SV40-CAT, containing the SV40 early promoter which has been shown to be expressed at high levels in skeletal muscle was used as a reference point for expression and the CAT basic promoterless vector was also included to detect background levels of expression. The SV40 promoter was chosen in preference to the CMV promoter as recent work in our laboratory had shown that the latter although stronly expressed in myoblast cultures does not give good expression in myotubes and hence it is unlikely to do so in mature muscle.In every transfection a plasmid bearing the β galactosidase reporter gene was co-transfected in order to calibrate for transfection efficiency.

The constructs were transfected in C2C12 myoblasts and these were allowed to differentiate for 4 days. Protein extracts of myotubes were assayed first for β-galactosidase activity. Subsequently, protein extracts corresponding to equal β-galactosidase were assayed for CAT activity so that a direct comparison of the relative transcriptional activities could be obtained. The results are shown in FIG. 7. All promoter fragments tested achieved levels of expression above the backround levels of the promoterless construct. The highest levels were obtained with the rabbit β myosin heavy chain bearing the myosin light chain enhancer. The transcriptional activity of this is combination was approximately 60% of that of the SV40 early promoter containing construct. These results indicated that the different plasmid constructs used were functional and that all promoter fragments tested were active although to different extents. However, C2C12 myotubes exhibit characteristics of embryonic muscle. Therefore, to obtain an assessment of the activity of these constructs in muscle fibres it was necessary to carry out experiments in vivo.

Transcriptional activity in fast and slow muscles. To assess the relative activities of the different constructs in vivo, 100 μg of test plasmid were injected in the tibialis anterior (TA) muscle of the mouse together with 60 μg of plasmid CH110. For the soleus muscle injections, 80 μg of test plasmid and 60 μg of CH110 were used. Ten days after injection, the muscles were removed and processed for CAT activity assays. FIG. 8 shows that expression was obtained with all constructs to different degrees in both TA and soleus muscles. The highest level of expression was obtained with the plasmid bearing the truncated rabbit β-cardiac myosin heavy chain fragment and the myosin light chain enhancer. The levels of expression obtained with this construct were approximately 80% of those of SV40-CAT the plasmid used as a reference. Varying levels of expression were obtained with the other plasmids, ranging from 69.4% of SV40-CAT expression for the βMHC-CAT plasmid containing the enhancer to 32.5% for the MLC-CAT plasmid in the tibialis anterior. Variation in expression levels was also observed in the soleus. A low level of expression (19.6% that of SV40-CAT) was observed with the βMHC-CAT construct. Given that this fragment is that of a slow MyHC this was an unexpected result. When the myosin light chain enhancer was included in the construct, expression rose approximately 4-fold. We are currently examining by immunocytochemistry, the muscle fibre specificity of this promoter and whether the presence of the enhancer results in changes in muscle fibre specificity or just in an increase in the levels of expression in the same muscle fibres.

Plasmid uptake by fast and slow muscles. The mechanism of uptake of plasmids by muscle fibres is so far poorly understood. One possibility is that it depends on fibre type. As a first step towards investigating this, the expression levels of the β-galactosidase containing plasmid, normalized in terms of protein content were compared in the two types of muscle. Table 2 shows that the activity of β-galactosidase is lower in the soleus. This could reflect differences in uptake by different muscle fibre types i.e. slow fibres take up less plasmid. However, it could also depend on the degree to which the two types of muscle are vascularised. The soleus muscle is highly vascularised. It is therefore possible that some of the injected plasmid is removed by the circulation before it can enter the muscle fibres. It is necessary to carry out further experiments in muscles with a mixture of fibre types in order to clarify this point.

TABLE 2
Comparison of β-galactosidase expression
in Tibialis Anterior and Soleus musles.

| Muscle | n | A₄₂₀/mg protein |
|---|---|---|
| TA | 48 | 0.267 ± 0.24 |
| Soleus | 48 | 0.218 ± 0.19 |

Table 2. Comparison of β-galactosidase expression in tibialis anterior and soleus muscles. The injected muscles were assayed (see Methods) for the purpose of standardisation of the CAT activities. The figures represent mean and standard deviation.

Analysis of expression of truncated β-myosin heavy chain constructs by fast and slow muscles. The tibialis anterior was injected with the constructs expressing either the truncated rabbit β-myosin heavy chain alone or the myosin heavy chain with the myosin light chain enhancer.

Adjacent transverse sections of muscles were stained for CAT and for myosin ATPase activity after alkaline preincubation. As FIG. 9a,b shows positive staining was obtained in fast and intermediate type fibres as well as some slow fibres with the truncated β-myosin heavy chain construct. In the construct bearing this promoter and the myosin light chain enhancer the staining was confined to the fast fibres (FIG. 9c,d).. This indicates that the presence an enhancer of a sarcomeric protein can result in a shift to a phenotype characteristic of the gene of origin in this type of construct. In these experiments; a lower degree of staining was seen in slow fibres. This may result from different degrees of plasmid uptake that reflect differences in membrane composition in different muscle fibre types.

This study presents data on levels of uptake and expression by fast and slow muscles of a number of different plasmid constructs where muscle specific promoter fragments are used to drive expression of the gene of interest. The results show that a range of expression levels can be obtained and that the presence of an enhancer in the construct greatly increases expression levels.

Several different types of vectors have so far been used for the introduction and expression of genes in muscle and they generally fall into two categories: retroviral and adenoviral vectors. These have been shown to produce adequate levels of expression of reporter genes¹¹ and dystrophin^{12,13}. However, only a relatively low percentage of positive fibres was achieved. In addition, the use of viral vectors could lead to recombinant events for example during muscle regeneration after injury, and the activation of endogenous genes.

Of the promoters tested, the rabbit truncated β-cardiac myosin heavy chain promoter gave an unexpected result ie its expression was lower in the soleus. Further experiments are under way to clarify this point. Rindt et al¹⁴ have used a 600 bp fragment from the mouse equivalent in transgenic experiments and reported¹⁵ that expression was dependent on the point of integration of the construct to the genome. It is possible that truncated promoters exhibit properties atypical of the isoform in which they belong, perhaps due to the removal of some regulatory elements.

Wolff et al¹⁶ have shown that when plasmids are introduced into muscle they are retained for long periods. In addition, retention by the muscle is episomatic, and this rules out side effects resulting from recombination events. The degree of uptake by muscle has been found to be age and sex-dependent³. In our study, the sex of mice (female) and their age (8-12 weeks) were comparable to the least favourable of their study, and yet a high degree of reporter gene expression was observed both with the SV40 early promoter construct and with some of those bearing muscle specific promoters. This may be due to the fact that the plasmids used in our study are to a large extent supercoiled and this would permit higher uptake by the muscle fibres. More recently it has also been shown in primary culture studies, that although a large proportion of the plasmid DNA taken up by myotubes is sequestered into cytoplasmic compartments, a large percentage of myotube nuclei take up the remaining plasmid.¹⁷

### MATERIALS AND METHODS

Plasmids. The promoter fragments were obtained as follows: rabbit β-cardiac from Dr. Patrick Umeda, University of Alabama, Birmingham Ala., USA human β-cardiac, Dr. Hans-Peter Vosberg, Max-Planck Institute, Bad Neuheim, Germany M-CK promoter/enhancer Dr. Steve Hauschka, University of Washington myosin light chain enhancer, Dr. Nadia Rosenthal Harvard University. Cloning into the CAT plasmid (Promega) was carried out by standard methods. Plasmid preparations were carried out using the Mega-Prep kits (Qiagen Germany). Plasmids obtained by these preparations are largely supercoiled.

Cell culture and tranfections. C2C12 cells were grown in DMEM (Gibco)containing 10% fetal calf serum (Gibco), 0.5% ampicillin(Sigma, UK) and 0.5% gentamycin(Sigma). The differentiation medium consisted of 5% horse serum(Gibco) in DMEM with antibiotics as above. Myoblasts were plated at 2×10⁵ cells/60 mm dish for transfections.

For each dish, 10 μg of test plasmid and 5 μg of the β-galactosidase containing plasmid were transfected, complexed with lipofectamine (Gibco). Transfections were carried out overnight. The cells were then switched to differentiation medium and allowed to differentiate for 4 days. They were then harvested and processed for CAT and β-galactosidase assays.

Injections in fast and slow muscles. 8-12 week old C57B1/10 mice (Olac, Oxford UK) were used. The injection mixture consisted of the test plasmid (100 μg) and 60 μg of the plasmid CH110 (Promega) which contains the β-galactosidase reported gene driven by the SV40 early promoter in PBS for tibialis anterior injections. In the soleus, 80 μg of test plasmid and 40 μg of CH110 were injected. The mice were sacrificed 10 day after injection and the muscles processed for enzyme assay or staining.

Protein assays. These were carried out by the bicinchoninic acid method¹⁹ using BSA as a standard.

β-galactosidase assays. The method described in Shambrook et al²⁰ was used with the exception that 20 ml of cell or muscle extract were used and the reactions were incubated overnight at 37° C.

CAT activity assays. These were carried out as in Ausubet et al²¹. Briefly, cells were washed three times in PBS and harvested in 100 μl of 0.25M Tris.HCl pH 7.6. Muscles were homogenized in 150 μl (TA) and 100 μl (soleus) 0.25M Tris pH 7.6, using a small pestle. The CAT assay reactions contained of 4 μl [¹⁴C] chloramphenicol and 20 μl 8 mM acetyl coenzymeA. The corresponding cell or muscle extract volumes were incubated for 2 hr at 37° C., extracted with 1 ml ethyl acetate and the residue was dissolved in 30 μl of ethyl acetate. The samples were loaded on TLC chromatographic plates and run for 2 hrs in chloroform methanol 19:1. The results were visualised by autoradiography. For measurements of the chloramphenicol conversion to its acetylated form, the spots were scraped and radioactivity was measured by liquid scintillation counting. The percentage conversion of chloramphenicol to its acetylated forms was then calculated. Immunocytochemistry. Muscles were injected and collected as described above and were embedded in melting isopentane. 12 μM cryosections were cut and adjacent sections were processed either for antibody staining or ATPase histochemistry. The CAT enzyme was detected with a sheep polyclonal antibody (Boehringer Mannheim, Germany). The immunostaining protocol was as follows:sections were fixed with 4% paraformaldehyde and 0.2% picric acid in PBS for 30 min, washed 3×10 min in PBS containing 0.1% BSA, treated with 3% H₂O₂ in PBS for 20 min, washed 3×10 min in PBS containing 0.1% BSA and incubated in a 1/50 dilution of primary antibody in 10% pre-immune goat serum at 4 C. overnight. Following washing 3×30 min with PBS/BSA solution, they were incubated in 1/200 dilution of anti sheep IgM-HRP(Fab fragments) for 2 hr at room temperature. Finally, the sections were developed using 3′3′-diaminobenzidine as a substrate.

Myosin ATPase histochemistry.Alkaline preincubation (pH 10.4) was carried out by the method of Guth and Samaha²² as modified by Hamalainen and Pette²³

### REFERENCES FOR EXAMPLE 2 ONLY

1.WOLFF, J. A., MALLONER R. W., WILLIAMS, P., CHONG, W., ASCADI, G., JANI, A., and FELGNER, P. L. (1990). Direct gene transfer into mouse muscle in vivo. Science, 247 1465-8.

2.HANSEN, e., FERNANDES, K., GOLDSPINK, G., BUTTERWORTH, P., UMEDA, P. K., CHANG, K. C. (1991) Strong expression of foreign genes following direct injection into fish muscle. FEBS Lett. 290, 73-6.

3.WELLS. D. J. and GOLDSPINK, G. (1992). Age and sex influence expression of plasmid DNA directly injected into mouse skeletal muscle. FEBS Lett 306, 203-5.

4.MILLER, G..STEINBRECHER, R. A.,MURDOCK, P. J., TUDDENHAM, E. G. D., LEE, C. A., PASI, K. J.,GOLDSPINK, G. (1995) Expression of factor VII by muscle cells in vitro and in vivo following direct gene transfer: Modelling gne therapy for haemophilia. Gene therapy 2, 736-742.

5. KEIR,S D., MITCHELL. W. J., FELDMAN, L., MARTIN, JR. (1995). Targeting and gene expression in spinal cord motoneurons following intramuscular inoculation of an HSV-1 vector. J.. Neurovirol. 1 259-267.

6. RIBOTA. M G Y, REVAH, F., PRADIER, L., LOQUET, I MALLET, J. PRIVAT,A. (1997). Prevention of motoneuron death by adenovirus-mediated neurotrophic factors. J. Neurosci. Res. 48 281-285.

7.CRIBBS, L. L., SHIMIZU, N., YOCKEY, C. E., LEVIN, J. E., JACOVIC, S., ZAK, R., UMEDA, P. K. (1989). Muscle-specific regulation of a transfected rabbit myosin heavy chain β gene promoter. J. Biol. Chem 264, 10672-10678.

8.DONOGHUE, M., ERNST, H., WENTWORTH, B., NADAL-GINARD, B., ROSENTHAL, N.(1988). A muscle-specific enhancer is located at the 3′ end of the myosin light-chain 1/3 gene locus. Genes Dev. 2,1779-1790.

9.VOSBERG., H-P. , HORSTMAN-HEROLD U., WETTSTE IN, A. (1992). The regulation of the human β myosin heavy-chain gene.Bas. Res. in Cardiol. 87 Supp. 1 161-173.

10.JAYNES, J. B., JOHNSON, J. J., BUSKIN, J. N., GARTSIDE, C. L., HAUSCHKA, S. D. (1988). The muscle creatine kinase gene is regulated by multiple upstream elements including a muscle specific enhancer. Mol. Cell. Biol. 8, 62-70

11.QUANTIN, M., PERRICAUDET, L. D., TAJBACHSH, S.,MANDEL, J. L. (1992). Adenovirus as an expression vector in mucle cells in vivo. PNAS 89, 2581-4.

12.ALLAMEDINE, H. S., QUANTIN, B., CARTAUD, A., DEHAUPAS, M., MANDEL, J. L., FARDEAU, M. (1994). Expression of a recombinant dystrophin in mdx mice using adenovirus vectors. Neuromusc.Disord. 4, 193-203.

13.DUX.,L.,COOPER, B. J., SEWRY, C. A and DUBOWITZ, V. (1993). Notechis scutatus venom increases the yield of proliferating muscle cells from biopsies of normal and dystrophic canine muscle—a possible source for myoblast transfer studies. Neuromuscular Disorders 3, 23-9.

14.RINDT, H., GULICK, J., KNOTTS, S., NEUMAN, J., ROBBINS, J. (1993). In vivo analysis of the murine β-myosin heavy chain gene promoter. J. Biol. Chem. 268, 5332-5338.

15.KNOTTS, S., RINDT, H., ROBBINS, J. (1995). Position independent expression and developmental regulation is directed by the β myosin heavy chain gene's upstream region in transgenic mice. Nucleic Acids. Res. 23, 3301-3309.

16.WOLFF, J. A.,LUDTKE, J. J., ASCADI, G., WILLIAMS, P. (1992). Long-term persistence of plasmid DNA and foreign gene expression in mouse muscle. Hum. Molec.Genetics 1 363-9.

17.DOWTY, M. E., WILLIAMS, P.,ZHANG, G., HAGSTROM, J. E., WOLLF, J. A. (1995). Plasmid DNA entry into postmitotic nuclei of primary rat myotubes. PNAS 92, 4572-6.

18.DAVIS, H. C.,DEMENEIX, B. A., QUANTIN, B., COULOMBE, J., WHALEN, R. G. (1993). Plasmid DNA is superior to viral vectors for direct gene transfer into adult mouse skeletal muscle. Hum. Gene Ther. 4, 733-40.

19.SMITH, P K., KROHN, R I., HERMANSON, G T., MALLIA, A K., GARTNER, F H. PROVENZANO, M D., FUJIMOTO, E K., GOEKE, N M. OLSON, B J., KLENK, D C. (1985). Measurement of protein using bicinchoninic acid. Anal. Biochem.150, 76-85.

20. SAMBROOK, J., FRITSCH, E. F.,MANIATIS, T.(1989). Molecular Cloning, A Laboratory Manual 2nd Edition Cold Spring Harbor Laboratory Press.

21.AUSUBET, F. M,BRENT, R.,KINGSTON, R. E., MOORE, D. D.,SEIDMAN, J. G. SMITH, J. A., STRUHL, K. (Eds). Current protocols in Molecular Biology, J. Wiley and Sons, New York, 1991.

22. GUTH, L. SAMATHA, F J. (1970). Procedure for the histochemical demonstration of actomyosin ATPase. Exp. Neurol. 28 365-367.

23.HAMALAINEN, N. PETTE, D.(1993). The histochemical profiles of fast fibre types IIB, IID, and IIA in skeletal muscles of mouse, rat and rabbit. J. Histochem. cytochem. 41, 733-743.

1. An expression cassette comprising, operably linked, (i) a myosin light chain enhancer, (ii) a promoter selected from a myosin heavy chain promoter and a viral promoter and (iii) a polynucleotide sequence of interest.

2. An expression cassette according to claim 1 wherein said myosin light chain enhancer is a myosin light chain 1/3 enhancer.

3. An expression cassette according to claim 1 or 2 wherein said myosin heavy chain promoter is a fish myosin heavy chain promoter.

4. An expression cassette according to claim 3 wherein said fish myosin heavy chain promoter is a carp FG2 myosin heavy chain promoter.

5. An expression cassette according to claim 1 or 2 wherein said myosin heavy chain promoter is a mammalian myosin heavy chain promoter.

6. An expression cassette according to claim 5 wherein said mammalian myosin heavy chain promoter is a truncated rabbit β-cardiac myosin heavy chain promoter.

7. An expression cassette according to any one of the preceding claims wherein said viral promoter is a cvtomegalovirus promoter or a herpes simplex virus promoter.

8. An expression cassette according to any one of the preceding claims wherein said polynucleotide sequence of interest encodes a polypeptide of therapeutic use.

9. An expression cassette according to claim 8 wherein said polypeptide is α-galactosidase.

10. An expression cassette according to any one of the preceding claims for use in delivering said polynucleotide sequence of interest to a eukaryotic cell.

11. An expression cassette according to claim 10 wherein said eukaryotic cell is a muscle cell of a bird, fish or mammal.

12. A nucleic acid vector comprising an expression cassette as defined in any one of the preceding claims.

13. A vector according to claim 12 further comprising avian, fish or mammalian genomic sequences flanking said expression cassette.

14. A vector according to claim 12 or 13 further comprising viral genomic sequences flanking said expression cassette.

15. A viral strain comprising an expression cassette as defined in any one of claims 1 to 11.

16. A method of producing a viral strain according to claim 15 which method comprises introducing an expression cassette as defined in any one of claims 1 to 11 into the genome of a virus.

17. A method of producing a viral strain according to claim 15 which method comprises introducing an expression cassette as defined in any one of claims 1 to 11 into the genome of a virus by homologous recombination between said genome and a vector as defined in claim 14.

18. An expression cassette according to any one of claims 1 to 11, a vector according to any one of claims 12 to 14 or a viral strain according to claim 15 for use in a method of treatment of the human or animal body.

19. An expression cassette according to any one of claims 1 to 11, a vector according to any one of claims 12 to 14 or a viral strain according to claim 15 for use in the treatment of Fabry disease.

20. Use of an expression cassette according any one of claims 1 to 11, a vector according to any one of claims 12 to 14 or a viral strain according to claim 15 in the treatment of Fabry disease.

21. A pharmaceutical composition comprising an expression cassette according to any one of claims 1 to 11, a vector according to any one of claims 12 to 14 or a viral strain according to claim 15 together with a pharmaceutically acceptable carrier or diluent.

22. A method of treatment of the human or animal body which method comprises administering an effective, non-toxic amount of a pharmaceutical composition according to claim 21 to a human or animal in need of such treatment.

23. A method according to claim 22 for the treatment of Fabry disease.

24. A method of effecting gene therapy in a human or animal which method comprises introducing an expression cassette according to any one of claims 1 to 11, a vector according to any one of claims 12 to 14 or a viral strain according to claim 15 into the cells of a human or animal in need of such therapy in an amount resulting in effective expression of a heterologous gene encoding a therapeutic polypeptide in said cells.

25. An expression cassette according to any one of claims 1 to 7 wherein said polynucleotide of interest encodes a polypeptide comprising at least one epitope.

26. An expression cassette according to claim 25 wherein said polypeptide is derived from a pathogenic organism.

27. A vector comprising an expression cassette according to claims 25 or 26.

28. A viral strain comprising an expression cassette according to claim 25 or 26.

29. An expression cassette according to claim 25 or 26, a vector according to claim 27 or a viral strain according to claim 28 for use in a method of vaccinating a bird, fish or mammal.

30. A vaccine comprising an expression cassette according to claim 25 or 26, a vector according to claim 27 or a viral strain according to claim 28 together with a pharmaceutically acceptable carrier or diluent.
